(19) United States
(12) Patent Application Publication
(10) Pub. No.: US 2006/0200113 A1
(43) Pub. Date: Sep. 7, 2006

(54) Liquid jet for glaucoma treatment

(76) Inventors: David Haffner, Mission Viejo, CA (US); Hosheng Tu, Newport Coast, CA (US); Gregory Smedley, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/353,854

(22) Filed: Feb. 13, 2006

Related U.S. Application Data
(63) Continuation-in-part of application No. 10/384,912, filed on Mar. 7, 2003.
(60) Provisional application No. 60/652,271, filed on Feb. 11, 2005.

(51) Int. Cl. A61B 18/18

(52) U.S. Cl. 606/6

(57) ABSTRACT

Methods of treating glaucoma are described including treatment of an eye with a liquid jet. The liquid jet can be generated by a laser-induced liquid jet instrument which is inserted through an incision in an eye. A stent can also be used, the stent having an inflow portion that is in fluid communication with an outflow portion. The stent can be inserted into the eye and transported from the incision through the anterior chamber of the eye toward the trabecular meshwork of the eye. In some embodiments, the stent can be advanced through the anterior chamber of the eye and provide fluid communication between the anterior chamber and Schlemm's canal. The method includes infusing fluid into an aqueous cavity and increasing the pressure of the fluid within the aqueous cavity.

### RELATED APPLICATIONS

This application is a continuation in part of U.S. application Ser. No. 10/384,912, filed Mar. 7, 2003, and claims benefit of U.S. Provisional Application No. 60/652,271, entitled “Fluid Infusion Means by Laser-induced Liquid Jet for Glaucoma Treatment,” filed Feb. 11, 2005, the entireties of both of which are hereby incorporated by reference.

### BACKGROUND OF THE INVENTIONS

1. Field of the Inventions

This disclosure relates to reducing intraocular pressure within the animal eye. More particularly, this disclosure relates to a treatment of glaucoma wherein aqueous humor is permitted to flow out of an anterior chamber of the eye through a surgically implanted pathway. Furthermore, this disclosure relates to directly dilating Schlemm's canal and/or aqueous collector channels by injecting fluid via laser-induced liquid jet through an opening into Schlemm's canal or through the implanted pathway of a stent.

2. Description of the Related Art

A human eye is a specialized sensory organ capable of light reception and is able to receive visual images. Aqueous humor is a transparent liquid that fills the region between the cornea, at the front of the eye, and the lens. A trabecular meshwork, located in an anterior chamber angle formed between the iris and the cornea, serves as a drainage channel for aqueous humor from the anterior chamber, which maintains a balanced pressure within the anterior chamber of the eye.

About two percent of people in the United States have glaucoma. Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucomas.

In glaucomas associated with an elevation in eye pressure (intraocular hypertension), the source of resistance to outflow is mainly in the trabecular meshwork. The tissue of the trabecular meshwork allows the aqueous humor (hereinafter referred to as “aqueous”) to enter Schlemm's canal, which then empties into aqueous collector channels in the posterior wall of Schlemm's canal and then into aqueous veins, which form the episcleral venous system. Aqueous is continuously secreted by a ciliary body around the lens, so there is a constant flow of aqueous from the ciliary body to the anterior chamber of the eye. Pressure within the eye is determined by a balance between the production of aqueous and its exit through the trabecular meshwork (major route) and uveoscleral outflow (minor route). The portion of the trabecular meshwork adjacent to Schlemm's canal (the juxtacanilicular meshwork) causes most of the resistance to aqueous outflow.

Glaucoma is broadly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous from the anterior chamber of the eye. Open-angle glaucoma is any glaucoma in which the exit of aqueous through the trabecular meshwork is diminished while the angle of the anterior chamber remains open. For most cases of open-angle glaucoma, the exact cause of diminished filtration is unknown. Primary open-angle glaucoma is the most common of the glaucomas, and is often asymptomatic in the early to moderately advanced stages of glaucoma. Patients may suffer substantial, irreversible vision loss prior to diagnosis and treatment. However, there are secondary open-angle glaucomas that may include edema or swelling of the trabecular spaces (e.g., from corticosteroid use), abnormal pigment dispersion, or diseases such as hyperthyroidism that produce vascular congestion.

All current therapies for glaucoma are directed toward decreasing intraocular pressure. Currently recognized categories of drug therapy for glaucoma include: (1) Miotics (e.g., pilocarpine, carbachol, and acetylcholinesterase inhibitors), (2) Sympathomimetics (e.g., epinephrine and dipivalylepinephxine), (3) Beta-blockers (e.g., betaxolol, levobunolol and timolol), (4) Carbonic anhydrase inhibitors (e.g., acetazolamide, methazolamide and ethoxzolamide), and (5) Prostaglandins (e.g., metabolite derivatives of arachindonic acid). Medical therapy includes topical ophthalmic drops or oral medications that reduce the production of aqueous or increase the outflow of aqueous. However, drug therapies for glaucoma are sometimes associated with significant side effects. The most frequent and perhaps most serious drawback to drug therapy is that patients, especially the elderly, often fail to correctly self-medicate. Such patients forget to take their medication at the appropriate times or else administer eye drops improperly, resulting in under- or overdosing. Because the effects of glaucoma are irreversible, when patients dose improperly, allowing ocular concentrations to drop below appropriate therapeutic levels, further permanent damage to vision occurs. Furthermore, current drug therapies are targeted to be deposited directly into the ciliary body where the aqueous is produced. And current therapies do not provide for a continuous slow-release of the drug. When drug therapy fails, surgical therapy is pursued.

Surgical therapy for open-angle glaucoma consists of laser trabeculoplasty, trabeculectomy, and implantation of aqueous shunts after failure of trabeculectomy or if trabeculectomy is unlikely to succeed. Trabeculectomy is a major surgery that is widely used and is augmented with topically applied anticancer drugs, such as 5-flurouracil or mitomycin-C to decrease scarring and increase the likelihood of surgical success.

Approximately 100,000 trabeculectomies are performed on Medicare-age patients per year in the United States. This number would likely increase if ocular morbidity associated with trabeculectomy could be decreased. The current morbidity associated with trabeculectomy consists of failure (10-15%); infection (a life long risk of 2-5%); choroidal hemorrhage, a severe internal hemorrhage from low intraocular pressure, resulting in visual loss (1%); cataract formation; and hypotony maculopathy (potentially reversible visual loss from low intraocular pressure). For these reasons, surgeons have tried for decades to develop a workable surgery for the trabecular meshwork.

The surgical techniques that have been tried and practiced are goniotomy/trabeculotomy and other mechanical disruptions of the trabecular meshwork, such as trabeculopuncture, goniophotoablation, laser trabecular ablation, and goniocurretage. These are all major operations and are briefly described below.

Goniotomy and trabeculotomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed due to cellular repair and fibrosis mechanisms and a process of “filling in.” Filling in is a detrimental effect of collapsing and closing in of the created opening in the trabecular meshwork. Once the created openings close, the pressure builds back up and the surgery fails.

Q-switched Neodynium (Nd) YAG lasers also have been investigated as an optically invasive trabeculopuncture technique for creating full-thickness holes in trabecular meshwork. However, the relatively small hole created by this trabeculopuncture technique exhibits a filling-in effect and fails.

Goniophotoablation is disclosed by Berlin in U.S. Pat. No. 4,846,172 and involves the use of an excimer laser to treat glaucoma by ablating the trabecular meshwork. This method did not succeed in a clinical trial. Hill et al. used an Erbium YAG laser to create full-thickness holes through trabecular meshwork (Hill et al., Lasers in Surgery and Medicine 11:341346, 1991). This laser trabecular ablation technique was investigated in a primate model and a limited human clinical trial at the University of California, Irvine. Although ocular morbidity was zero in both trials, success rates did not warrant further human trials. Failure was again from filling in of surgically created defects in the trabecular meshwork by repair mechanisms. Neither of these is a viable surgical technique for the treatment of glaucoma.

Goniocurretage is an “ab interno” (from the inside), mechanically disruptive technique that uses an instrument similar to a cyclodialysis spatula with a microcurrette at the tip. Initial results were similar to trabeculotomy: it failed due to repair mechanisms and a process of filling in.

Although trabeculectomy is the most commonly performed filtering surgery, viscocanalostomy (VC) and nonpenetrating trabeculectomy (NPT) are two new variations of filtering surgery. These are “ab externo” (from the outside), major ocular procedures in which Schlemm's canal is surgically exposed by making a large and very deep scleral flap. In the VC procedure, Schlemm's canal is cannulated and viscoelastic substance injected (which dilates Schlemm's canal and the aqueous collector channels). In the NPT procedure, the inner wall of Schlemm's canal is stripped off after surgically exposing the canal.

Trabeculectomy, VC, and NPT involve the formation of an opening or hole under the conjunctiva and scleral flap into the anterior chamber, such that aqueous is drained onto the surface of the eye or into the tissues located within the lateral wall of the eye. These surgical operations are major procedures with significant ocular morbidity. When trabeculectomy, VC, and NPT are thought to have a low chance for success, a number of implantable drainage devices have been used to ensure that the desired filtration and outflow of aqueous through the surgical opening will continue. The risk of placing a glaucoma drainage device also includes hemorrhage, infection, and diplopia (double vision).

All of the above embodiments and variations thereof have numerous disadvantages and moderate success rates. They involve substantial trauma to the eye and require great surgical skill in creating a hole through the full thickness of the sclera into the subconjunctival space. The procedures are generally performed in an operating room and involve a prolonged recovery time for vision. The complications of existing filtration surgery have prompted ophthalmic surgeons to find other approaches to lowering intraocular pressure.

Because the trabecular meshwork and juxtacanilicular tissue together provide the majority of resistance to the outflow of aqueous, they are logical targets for surgical removal in the treatment of open-angle glaucoma. In addition, minimal amounts of tissue need be altered and existing physiologic outflow pathways can be utilized.

As reported in Arch. Ophthalm. (2000) 118:412, glaucoma remains a leading cause of blindness, and filtration surgery remains an effective, important option in controlling glaucoma. However, modifying existing filtering surgery techniques in any profound way to increase their effectiveness appears to have reached a dead end.

### SUMMARY OF THE INVENTION

What is needed, is an extended, site-specific treatment method for placing a hollow trabecular microstent ab interno for diverting aqueous humor in an eye from the anterior chamber into Schlemm's canal. In some aspect of the present disclosure, a method is provided for injecting laser-induced liquid, optionally through the common hollow lumen of the microstent, to therapeutically dilate Schlemm's canal and the aqueous collector channels.

A device and methods are provided for improved treatment of intraocular pressure due to glaucoma. A hollow trabecular microstent is adapted for implantation within a trabecular meshwork of an eye such that aqueous humor flows controllably from an anterior chamber of the eye to Schlemm's canal, bypassing the trabecular meshwork. The trabecular microstent comprises a quantity of pharmaceuticals effective in treating glaucoma, which are controllably released from the device into cells of the trabecular meshwork and/or Schlemm's canal. Depending upon the specific treatment contemplated, pharmaceuticals may be utilized in conjunction with the trabecular microstent such that aqueous flow either increases or decreases as desired. Placement of the trabecular microstent within the eye and incorporation, and eventual release, of a proven pharmaceutical glaucoma therapy will reduce, inhibit or slow the effects of glaucoma.

One aspect of the disclosure provides an axisymmetric trabecular microstent that is implantable within an eye. The microstent comprises an inlet section containing at least one lumen and one inlet opening, an outlet section having at least one lumen that connects to at least one outlet opening. In some aspects of the present disclosure, the microstent further comprises a flow-restricting member within the lumen that is configured to partially prevent back flow from passing through the flow-restricting member. The microstent further comprises a middle section that is fixedly attached to the outlet section having at least one lumen in fluid communication with the lumen of the outlet section. The middle section is fixedly attached to the inlet section and the lumen within the middle section is in fluid communication with the lumen of the inlet section. The device is configured to permit fluid entering the lumen of the inlet section to pass through the flow-restricting member, enter the lumen of the middle section, pass into the lumen of the outlet section, and then exit the outlet section.

Another aspect of the disclosure provides a method of treating glaucoma. The method comprises providing fluid through the lumen of the microstent to therapeutically dilate the aqueous cavity. The term “aqueous cavity” herein refers to any one or more of the aqueous cavities or passageways in which aqueous humor is collected or passes, and includes, without limitation, Schlemm's canal, aqueous collector channels, aqueous veins, and episcleral veins. In one embodiment, the fluid contains therapeutic substance, including pharmaceuticals, genes, growth factors, enzymes and like. In another embodiment, the fluid contains sterile saline, viscoelastic, or the like. The mode of fluid injection may be a pulsed mode, an intermittent mode or a programmed mode. In one aspect, the pressure of the fluid therapy is effective to cause therapeutic effects on the tissue of the aqueous cavity. In another aspect, the fluid pressure is effective to cause the dilation of the aqueous cavity beyond the tissue elastic yield point for permanent (i.e., plastic) deformation. In other embodiment, the fluid is at an elevated pressure effective to cause plastic deformation for at least a portion of the aqueous cavity. In still another embodiment, the pressurized fluid is generated in situ by a laser-induced liquid jet system.

Another aspect of the disclosure provides an apparatus for implanting a trabecular microstent within an eye and dilating the aqueous cavity. The apparatus comprises a syringe portion and a cannula portion that has proximal and distal ends. The proximal end of the cannula portion is attached to the syringe portion. The cannula portion further comprises a first lumen and at least one irrigating hole disposed between the proximal and distal ends of the cannula portion. The irrigating hole is in fluid communication with the lumen. The apparatus further includes a holder including a second lumen for holding the trabecular microstent. A distal end of the second lumen opens to the distal end of the cannula portion, and a proximal end of the second lumen is separated from the first lumen of the cannula portion. The holder holds the trabecular microstent during implantation of the device within the eye, and the holder releases the trabecular microstent when a practitioner activates deployment of the device.

Another aspect of the disclosure provides a method of implanting a trabecular microstent within an eye. The method comprises creating a first incision in a cornea on a first side of the eye, wherein the first incision passes through the cornea into an anterior chamber of the eye. The method further comprises passing an incising device through the first incision and moving a distal end of the incising device across the anterior chamber to a trabecular meshwork residing on a second side of the eye, and using the incising device to create a second incision. The second incision is in the trabecular meshwork, passing from the anterior chamber through the trabecular meshwork into a Schlemm's canal. The method further comprises inserting the trabecular microstent into a distal space of a delivery applicator. The delivery applicator comprises a cannula portion having a distal end and a proximal end attached to a syringe portion. The cannula portion has at least one lumen and at least one irrigating hole disposed between proximal and distal ends of the cannula portion. The irrigating hole is in fluid communication with the lumen. The distal space comprises a holder that holds the trabecular microstent during delivery and releases the trabecular microstent when a practitioner activates deployment of the device. The method further comprises advancing the cannula portion and the trabecular microstent through the first incision, across the anterior chamber and into the second incision, wherein an outlet section of the trabecular microstent is implanted into Schlemm's canal while an inlet section of the trabecular microstent remains in fluid communication with the anterior chamber. The method still further comprises releasing the trabecular microstent from the holder of the delivery applicator.

One aspect of the disclosure includes a method of treating glaucoma that includes inserting a stent through an incision in an eye, the stent having an inflow portion that is in fluid communication with an outflow portion of the stent, and transporting the stent from the incision through the anterior chamber of the eye to an aqueous cavity of the eye such that the inflow portion of the stent is positioned in the anterior chamber and the outflow portion of the stent is positioned at the aqueous cavity. The method also includes infusing fluid from the inflow portion to the outflow portion of the stent.

Some embodiments further include closing the incision, leaving the stent in the eye such that the inflow portion of the stent is positioned in the anterior chamber of the eye and the outflow portion of the stent is positioned in Schlemm's canal.

Some embodiments further include positioning the stent such that fluid communicating from the inflow portion to the outflow portion of the stent bypasses the trabecular meshwork of the eye.

In some embodiments fluid is infused through a lumen of the stent into an aqueous cavity, which may be Schlemm's canal. In other embodiments the aqueous cavity can be an aqueous collector channel, aqueous veins, or episcleral veins. In some embodiments, the infusing further comprises injecting the fluid in at least one of a pulsed mode, an intermittent mode, and a programmed mode.

In some embodiments the infusing of fluid is at a pressure sufficient to cause plastic deformation of at least a portion of the aqueous cavity. In some embodiments, the fluid can be at least one of a salt solution or viscoelastic.

In some embodiments, the infusing further comprises coupling the inflow portion of the stent with a fluid delivery element that transmits the fluid to the stent. In one embodiment, the coupling comprises securing a screw thread arrangement of the fluid delivery element with a receiving thread arrangement of the stent.

In certain preferred arrangements, the fluid comprises a therapeutic substance such as a pharmaceutical, a gene, a growth factor, and/or an enzyme. In some preferred arrangements, the fluid comprises a therapeutic substance such as an antiglaucoma drug, a beta-adrenergic antagonist, a TGF-beta compound, and/or an antibiotic.

Some embodiments provide that a temperature of the fluid is raised sufficiently to enhance the plastic deformation. And some embodiments provide that a pH of the fluid is adjusted sufficiently to enhance the plastic deformation. In some arrangements the method further includes vibrating a tissue of the eye.

One aspect of the disclosure includes a method of treating glaucoma, including inserting a stent through an incision in an eye, the stent having an inflow portion that is in fluid communication with an outflow portion of the stent. The method further includes positioning the stent such that the inflow portion of the stent is positioned in the anterior chamber of the eye and the outflow portion of the stent is positioned at an aqueous cavity and infusing fluid from the inflow portion to the outflow portion of the stent.

In one embodiment disclosed herein, a method of treating glaucoma includes inserting a jet instrument into the anterior chamber of an eye through an incision in the eye and advancing the instrument through the anterior chamber toward the trabecular meshwork of the eye. The method further includes generating with the instrument at least one of a liquid jet, plasma jet, and gas jet and creating a hole in the trabecular meshwork with at least one of said liquid jet, plasma jet, and gas jet. The method can further include contacting the trabecular meshwork with the jet instrument before creating the hole. The liquid jet that is generated can use a laser and the liquid jet can include water. In some embodiments, the liquid used to form the liquid jet includes aqueous humor. The method can further include administering a therapeutic agent through the jet instrument. As used herein, the term “jet instrument” is intended to have its ordinary meaning, which includes, without limitation, an instrument that creates, transmits, or otherwise facilitates a fluid jet, including a liquid jet, plasma jet, and gas jet. In some embodiments, the jet instrument can create a laser-induced jet.

In another embodiment, a method of treating glaucoma is disclosed that includes inserting a distal portion of a jet instrument into an aqueous cavity of an eye and generating at least one of a liquid jet, plasma jet, and gas jet with said instrument. The method further includes introducing said jet into said aqueous cavity thereby increasing a fluid pressure in the aqueous cavity of the eye. The aqueous cavity can include at least one of Schlemm's canal of the eye, an aqueous collector channel, an aqueous vein, and an episcleral vein of the eye. The method can further include engaging the distal portion of the instrument with a glaucoma implant, and the implant can be configured to conduct fluid away from the anterior chamber. The method can further include advancing said glaucoma implant with the distal portion of the instrument. The method can also include directing at least one of said liquid jet, plasma jet, and gas jet through a lumen of said implant, said implant being configured to conduct fluid away from the anterior chamber. In another embodiment, the method can also include elevating said pressure to a level sufficient to cause plastic deformation of the aqueous cavity. The liquid jet can include water, and the liquid used to form the liquid jet can include aqueous humor. In yet another embodiment, the method can include administering a therapeutic agent through the jet instrument.

### BRIEF DESCRIPTION OF THE DRAWINGS

FIG. 1 is a coronal, cross-sectional view of an eye.

FIG. 2 is an enlarged cross-sectional view of an anterior chamber angle of the eye of FIG. 1.

FIG. 3 is an oblique elevation view of one embodiment of a trabecular microstent.

FIG. 4 is a detailed view of the proximal section of the microstent of FIG. 3.

FIG. 5 is an applicator for delivering a microstent and infusing fluid for therapeutic treatment.

FIG. 6 is an enlarged, cross-sectional view of a preferred method of implanting a trabecular microstent within an eye.

FIG. 7 shows an embodiment of a distal portion of a jet instrument.

FIG. 8 shows a fiber optic probe system for providing a jet instrument in an eye.

### DETAILED DESCRIPTION OF THE PREFERRED EMBODIMENT

The preferred embodiments of the present disclosure described below relate particularly to surgical and therapeutic treatment of glaucoma through reduction of intraocular pressure. While the description sets forth various embodiment specific details, it will be appreciated that the description is illustrative only and should not be construed in any way as limiting the disclosure. Furthermore, various applications of the disclosure, and modifications thereto, which may occur to those who are skilled in the art, are also encompassed by the general concepts described below.

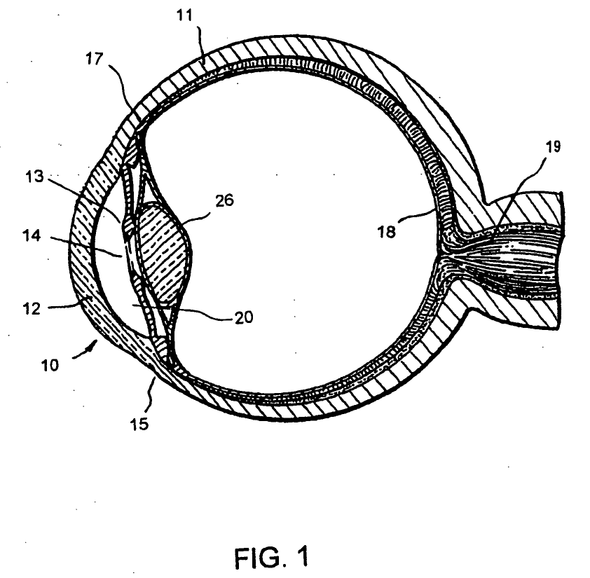

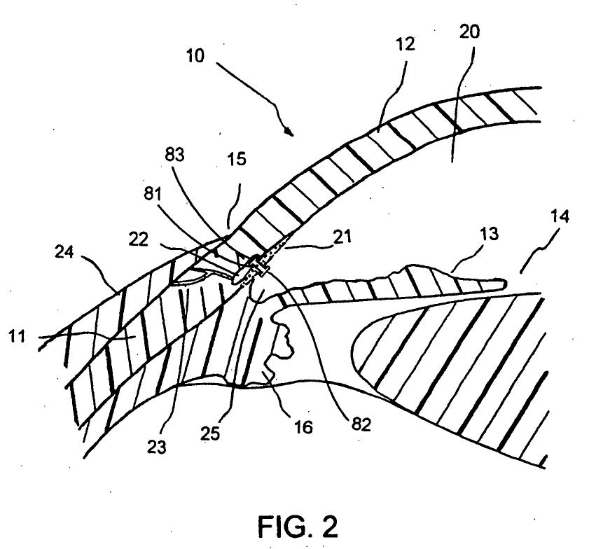

FIG. 1 is a cross-sectional view of an eye 10, while FIG. 2 is a close-up view showing the relative anatomical locations of a trabecular meshwork 21, an anterior chamber 20, and a Schlemm's canal 22. A sclera 11 is a thick collagenous tissue that covers the entire eye 10 except a portion that is covered by a cornea 12. The cornea 12 is a thin transparent tissue that focuses and transmits light into the eye and through a pupil 14, which is a circular hole in the center of an iris 13 (colored portion of the eye). The cornea 12 merges into the sclera 1I1 at a juncture referred to as a limbus 15. A ciliary body 16 extends along the interior of the sclera 11 and is coextensive with a choroid 17. The choroid 17 is a vascular layer of the eye 10, located between the sclera 11 and a retina 18. An optic nerve 19 transmits visual information to the brain and is the anatomic structure that is progressively destroyed by glaucoma.

The anterior chamber 20 of the eye 10, which is bound anteriorly by the cornea 12 and posteriorly by the iris 13 and a lens 26, is filled with aqueous humor (“aqueous”). Aqueous is produced primarily by the ciliary body 16, then moves anteriorly through the pupil 14 and reaches an anterior chamber angle 25, formed between the iris 13 and the cornea 12. In a normal eye, aqueous is removed from the anterior chamber 20 through the trabecular meshwork 21. Aqueous passes through the trabecular meshwork 21 into Schlemm's canal 22 and thereafter through a plurality of aqueous veins 23, which merge with blood-carrying veins, and into systemic venous circulation. Intraocular pressure is maintained by an intricate balance between secretion and outflow of aqueous in the manner described above. Glaucoma is, in most cases, characterized by an excessive buildup of aqueous in the anterior chamber 20, which leads to an increase in intraocular pressure. Fluids are relatively incompressible, and thus intraocular pressure is distributed relatively uniformly throughout the eye 10.

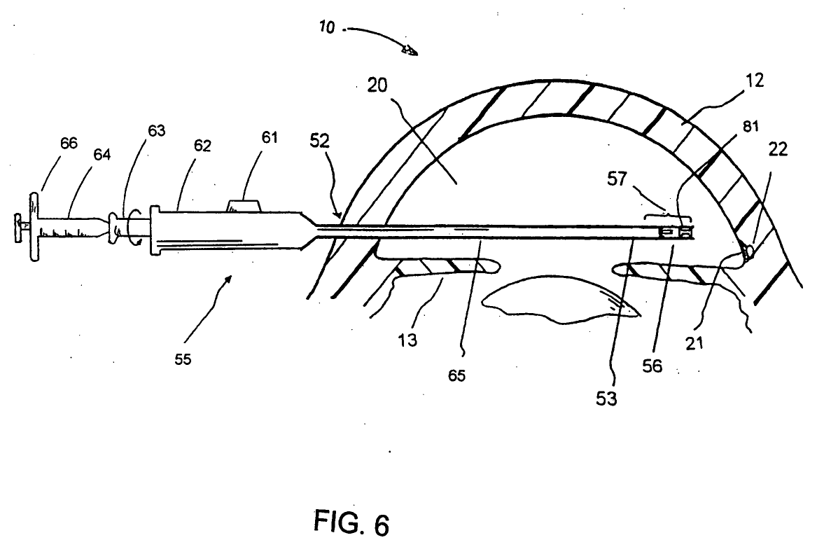

As shown in FIG. 2, the trabecular meshwork 21 is adjacent to a small portion of the sclera 11. Exterior to the sclera 11 is a conjunctiva 24. Traditional procedures that create a hole or opening for implanting a device through the tissues of the conjunctiva 24 and sclera 11 involve extensive surgery, as compared to surgery for implanting a device, as described herein, which ultimately resides entirely within the confines of the sclera 11 and cornea 12. A microstent, or implant, 81 is shown placed through trabecular meshwork 21 having a distal portion 83 disposed within Schlemm's canal 22 and a proximal portion 82 disposed within the anterior chamber 20 of the eye 10. FIG. 6 generally illustrates the use of one embodiment of a trabecular microstent 81 for establishing an outflow pathway, passing through the trabecular meshwork 21, which is discussed in greater detail below.

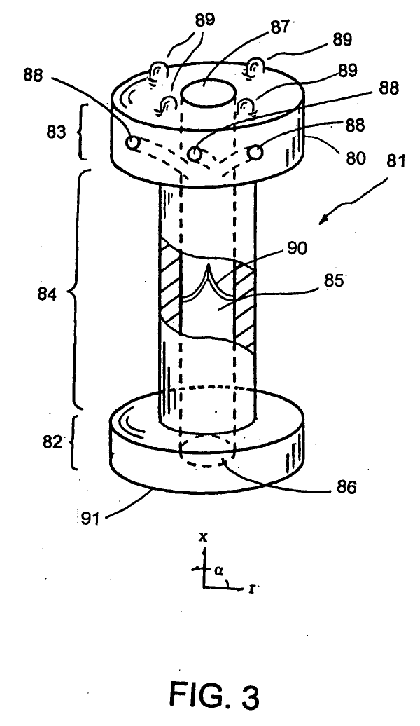

FIG. 3 illustrates an embodiment of a hollow trabecular microstent, stent, or implant, 81, which facilitates the outflow of aqueous from the anterior chamber 20 into Schlemm's canal 22, and subsequently into the aqueous collectors and the aqueous veins so that intraocular pressure is reduced. In the illustrated embodiment, the trabecular microstent 81 comprises an inlet section 82, having an inlet opening 86, a middle section 84, and an outlet section 83 having at least one opening 87, 88. The middle section 84 may be an extension of, or may be coextensive or confluent with, the inlet section 82. The device 81 comprises at least one lumen 85 within section 84, which is in fluid communication with the inlet opening 86 and the outlet opening 87, 88, thereby facilitating transfer of aqueous through the device 81. In one aspect, the outlet side openings 88, each of which is in fluid communication with the lumen 85 for transmission of aqueous, are arranged spaced apart around the circumferential periphery 80 of the outlet section 83. In another aspect, the outlet openings 88 are located and configured to enable jet-like infusing fluid impinging any specific region of Schlemm's canal tissue suitably for tissue stimulation. Several designs and shapes of stents or implants can be used in connection with the principles of this disclosure and as are known to those of ordinary skill in the art. For example, U.S. patent application Ser. No. 11/083,713 filed, Mar. 18, 2005, the entirety of which is hereby incorporated by reference herein, discloses various embodiments of stents or implants that can be deployed according to the principles disclosed herein.

As will be apparent to a person skilled in the art, the lumen 85 and the remaining body of the outlet section 83 may have a cross-sectional shape that is oval, circular, or other appropriate shape. Preferably, the middle section 84 has a length that is roughly equal to a thickness of the trabecular meshwork 21, which typically ranges between about 100 μm and about 300 μm.

To further stent or open Schlemm's canal after implanting a device 81, a plurality of elevated (that is, protruding axially) supports or pillars 89 can be located at the distal-most end of the outlet section 83 sized and configured for allowing media (for example, aqueous, liquid, balanced salt solution, viscoelastic fluid, therapeutic agents, or the like) to be transported freely.

The microstent 81 may further comprises a flow-restricting member 90, which is tightly retained within a lumen 85. The flow-restricting member 90 serves to selectively restrict at least one component in blood from moving retrograde, i.e., from the outlet section 83 into the anterior chamber 20 of the eye 10. Alternatively, the flow-restricting member 90 may be situated in any location within the device 81 such that blood flow is restricted from retrograde motion. The flow-restricting member 90 is sized and configured for maintaining the pressure of the infused fluid within the aqueous cavity for a suitable period of time. The flow-restricting member 90 may, in other embodiments, be a filter made of a material selected from the following filter materials: expanded polytetrafluoroethylene, cellulose, ceramic, glass, Nylon, plastic, and fluorinated material such as polyvinylidene fluoride (“PVDF”) (trade name: KYNAR®, by DuPont).

The trabecular microstent 81 may be made by molding, thermo-forming, or other micro-machining techniques. The trabecular microstent 81 preferably comprises a biocompatible material such that inflammation arising due to irritation between the outer surface of the device 81 and the surrounding tissue is minimized. Biocompatible materials which may be used for the device 81 preferably include, but are not limited to, titanium, stainless steel, medical grade silicone, e.g., SILASTIC™, available from Dow Coming Corporation of Midland, Mich.; and polyurethane, e.g., PELLETHANE™, also available from Dow Coming Corporation. In other embodiments, the device 81 may comprise other types of biocompatible material, such as, by way of example, polyvinyl alcohol, polyvinyl pyrolidone, collagen, heparinized collagen, polytetrafluoroethylene, expanded polytetrafluoroethylene, fluorinated polymer, fluorinated elastomer, flexible fused silica, polyolefin, polyester, polysilicon, and/or a mixture of the aforementioned biocompatible materials, and the like. In another aspect, the microstent is made of a biodegradable material selected from a group consisting of poly(lactic acid), polyethylene-vinyl acetate, poly(lactic-co-glycolic acid), poly(D,L-lactide), poly(D,L-lactide-co-trimethylene carbonate), poly(caprolactone), poly(glycolic acid), and copolymer thereof.

In still other embodiments, composite biocompatible material may be used, wherein a surface material may be used in addition to one or more of the aforementioned materials. For example, such a surface material may include polytetrafluoroethylene (PTFE) (such as TEFLON™), polyimide, hydrogel, heparin, therapeutic drugs (such as beta-adrenergic antagonists, TGF-beta, and other anti-glaucoma drugs, or antibiotics), and the like.

As is well known in the art, a device coated or loaded with a slow-release substance can have prolonged effects on local tissue surrounding the device. The slow-release delivery can be designed such that an effective amount of substance is released over a desired duration. “Substance,” as used herein, is intended to carry its ordinary meaning and includes, without limitation, any therapeutic or active drug that can stop, mitigate, slow-down or reverse undesired disease processes.

In one embodiment, the device 81 may be made of a biodegradable (also including bioerodible) material admixed with a substance for substance slow-release into ocular tissues. In another embodiment, polymer films may function as substance containing release devices whereby the polymer films may be coupled or secured to the device 81. The polymer films may be designed to permit the controlled release of the substance at a chosen rate and for a selected duration, which may also be episodic or periodic. Such polymer films may be synthesized such that the substance is bound to the surface or resides within a pore in the film so that the substance is relatively protected from enzymatic attack. The polymer films may also be modified to alter their hydrophilicity, hydrophobicity and vulnerability to platelet adhesion and enzymatic attack.

The device 81 may be used for a direct release of pharmaceutical preparations into ocular tissues. As discussed above, the pharmaceuticals may be compounded within the device 81 or form a coating on the device 81. Any known drug therapy for glaucoma may be utilized.

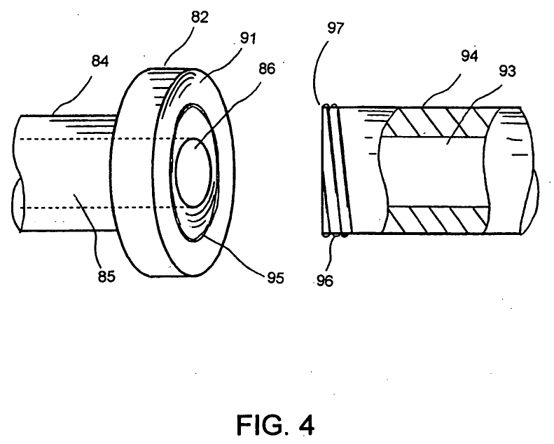

FIG. 4 shows a detailed view of the proximal section 82 of the microstent 81 of FIG. 3. In some aspect, the proximal section 82 has a bottom peripheral surface 91 that is about perpendicular to the lumen 85 of the microstent 81. A receiving thread arrangement 95 is appropriately located on the peripheral surface 91. The receiving thread arrangement 95 is sized and configured to releasably receive a screw thread arrangement 96 for coupling together, wherein the screw thread arrangement 96 is disposed at the distal end 97 of a fluid delivery element 94 which has a lumen 93 for transporting the infusing fluid into the aqueous cavity for therapeutic purposes. The coupling of the receiving thread arrangement 95 and the screw thread arrangement 96 makes the fluid infusion through the lumen 85 leak-proof enabling pressurization of the aqueous cavity.

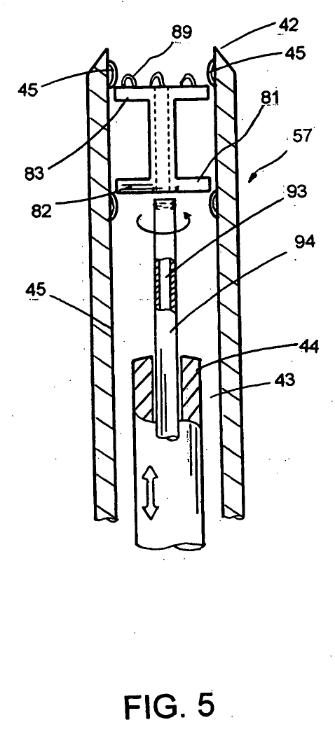

FIG. 5 shows a distal portion 57 of an applicator for delivering a microstent 81 and infusing fluid for therapeutic treatment. The distal portion 57 prefereably comprises a distal cutting means 42 sharp enough for creating an incision on the cornea and also creating an opening on trabecular meshwork 21 for stent placement. The microstent 81 may be axisymmetric and can be positioned within the lumen 43 of the applicator and is preferably retained by a plurality of stent retaining members 45. The microstent 81 is deployed from the applicator once the distal section 83 passes beyond the edge of the trabecular meshwork 21. In one aspect, the stent deployment is facilitated by a plunger-type deployment mechanism 44 with an associated deployment actuator 61 mounted on the handle 62 of the applicator (see FIG. 6). Other methods of deployment can also be used. For example, U.S. patent application Ser. No. 10/231342, filed Aug. 28, 2002, the entirety of which is hereby incorporated herein by reference, discloses other deployment mechanisms and methods that can be used in accordance with the principles disclosed herein.

The microstent 81 may be releasably coupled with a fluid delivery element 94 at any convenient time during the procedures. In one aspect, the screw-unscrew coupling steps between the microstent 81 and the fluid delivery element 94 is carried out by suitably rotating the fluid delivery element 94 with reference to the stent receiving thread arrangement 95, wherein the associated rotating mechanism 63 is located at the handle 62 of the applicator.

As will be appreciated by those of ordinary skill in the art, the device 81 may advantageously be practiced with a variety of sizes and shapes without departing from the scope of the disclosure. Depending upon the distance between the anterior chamber 20 and the drainage vessel (e.g., a vein) contemplated, the devices 81 may have a length ranging from about 0.05 centimeters to over about 1 centimeter. Preferably, the device 81 has an outside diameter ranging between about 30 μm and about 500 μm, with the lumen 85 having diameters ranging between about 20 μm and about 250 μm, respectively. In addition, the device 81 may have a plurality of lumens to facilitate transmission of multiple flows of aqueous or infusing fluid.

One preferred method for increasing aqueous outflow in the eye 10 of a patient, to reduce intraocular pressure therein, comprises bypassing the trabecular meshwork 21. In operation, the middle section 84 of the device 81 is advantageously placed across the trabecular meshwork 21 through a slit or opening. This opening can be created by use of a laser, a knife, thermal energy (radiofrequency, ultrasound, and microwave), cryogenic energy, or other surgical cutting instrument. The opening may advantageously be substantially horizontal, i.e., extending longitudinally in the same direction as the circumference of the limbus 15 (FIG. 2). Other methods for creating an opening may also be used, as are known by those of ordinary skill in the art. The opening may advantageously be oriented at any angle, relative to the circumference of the limbus 15, that is appropriate for inserting the device 81 through the trabecular meshwork 21 and into Schlemm's canal 22 or other outflow pathway, as will be apparent to those skilled in the art. Furthermore, the outlet section 83 may be positioned into fluid collection channels of the natural outflow pathways. Such natural outflow pathways include Schlemm's canal 22, aqueous collector channels, aqueous veins, and episcleral veins.

FIG. 6 generally illustrates a preferred method by which the trabecular microstent 81 is implanted within the eye 10. In the illustrated method, a delivery applicator is provided, which preferably comprises a syringe portion 64 and a cannula portion 65, which contains at least one lumen 43 in fluid communication with the fluid supply 66. The cannula portion 65 preferably has a size of about 30 gauges. However, in other embodiments, the cannula portion 65 may have a size ranging between about 16 gauges and about 40 gauges. A holder 56 at the distal portion 57 of the cannula portion 65 for holding the device 81 may advantageously comprise a lumen, a sheath, a clamp, tongs, a space, and the like.

In the method illustrated in FIG. 6, the device 81 is placed into the lumen 43 of the delivery applicator and then advanced to a desired implantation site within the eye 10. The delivery applicator holds the device 81 securely during delivery and releases it when the practitioner initiates deployment actuator 61 of the applicator.

In a preferred embodiment of trabecular meshwork surgery, a patient is placed in a supine position, prepped, draped, and appropriately anesthetized. A small incision 52 is then made through the cornea 12, for example, with a self-trephining applicator. The incision 52 preferably has a surface length less than about 1.0 millimeter in length and may advantageously be self-sealing. Through the incision 52, the trabecular meshwork 21 is accessed, wherein an incision is made with a cutting means 42 to form a hole in the trabecular meshwork 21 for stent placement. The hole in the trabecular meshwork can also be created with a tip having thermal energy or cryogenic energy. After the device 81 is appropriately implanted, the applicator is withdrawn and the trabecular meshwork surgery is concluded.

In some aspects of the present disclosure, a method is provided for expanding or attenuating the capacity of the existing canal outflow system (also known as the “aqueous cavity”). This system could have become constricted or blocked due to age or other factors associated with glaucoma. In one aspect, a tight fluid coupling is established between an external pressured fluid source 66 and Schlemm's canal 22 through a microstent 81. It is also advantageous to connect the external pressurized fluid source through a removable instrument (for example, a temporary applicator, catheter, cannula, or tubing) to Schlemm's canal ab interno for applying the fluid infusion therapy.

Once the fluid coupling is established, the pressure in the canal is raised by injecting fluid or fluid with therapeutic substances. In some aspect of the present disclosure, a method is provided of treating glaucoma including infusing fluid into aqueous cavity from an anterior chamber end of a stent, wherein the fluid is at an elevated pressure above a baseline pressure of the aqueous cavity. The method further comprises placing a hollow trabecular microstent bypassing the trabecular meshwork, wherein the fluid is infused from the anterior chamber through a lumen of the microstent. The mode of fluid injection is selected from a group consisting of a pulsed mode, an intermittent mode, a programmed mode, or combination thereof. In one aspect, the pressure of the fluid therapy is effective to cause therapeutic effects on the tissue of the aqueous cavity. In another aspect, the fluid pressure is effective to cause the dilation of the aqueous cavity beyond the tissue elastic yield point for plastic permanent deformation. In other embodiments, the fluid is at an elevated pressure effective to cause plastic deformation for at least a portion of the aqueous cavity. Other methods can be used, as are known by those of ordinary skill in the art and as described in U.S. application Ser. No. 10/384,912, filed Mar. 7, 2003, the entirety of which is hereby incorporated by reference herein.

The fluid may be a salt solution such as Balanced Salt Solution, a viscoelastic (such as Healon), any other suitable viscous or non-viscous liquid, or suitable liquid loaded with drug at a concentration suitable for therapeutic purposes without causing safety concerns. A combination of liquids may also be used. The pressure is raised at an appropriate rate of rise to an appropriate level and for an appropriate length of time, as determined through development studies, to provide for the expansion of the outflow structures and/or a clearing of any blockages within them. The procedure can be augmented with other aids to enhance its effectiveness. These aids may include heat, vibration (sonic or ultrasonic), pulsation of a pressure front, pH, drugs, etc. It is intended that the aqueous cavity be expanded (attenuation or tissue stimulation) by this procedure resulting in an increased capacity for inflow and outflow of Schlemm's canal.

In some aspects of the present disclosure, a method is provided for using a removable applicator, catheter, cannula, or tubing that is placed ab interno through the trabecular meshwork into the aqueous cavity of an eye adapted for infusing therapeutic liquid into the aqueous cavity.

In some aspects of the present disclosure, a method of treating glaucoma is provided. The method can include providing at least one pharmaceutical substance incorporated into a trabecular microstent and implanting the microstent within a trabecular meshwork of an eye such that a first end of the microstent is positioned in an anterior chamber of the eye while a second end is positioned in a Schlemm's canal. The first and second ends of the microstent can establish a fluid communication between the anterior chamber and the Schlemm's canal and allow the microstent to release a quantity of the pharmaceutical substance into the eye. In one embodiment, the method further comprises a step of infusing fluid into the Schlemm's canal from the anterior chamber through a lumen of the microstent, wherein the fluid is at an elevated pressure above a baseline pressure of the Schlemm's canal.

High-pressure, directed microjets can selectively dissect soft tissues while keeping the surrounding structure intact. Microjets can be used in liver surgery, in vascular eye surgery, and in conventional neurosurgery. However, the conventional water jet can lead to water accumulation and elevated pressure without proper venting. Sometimes water bubbles can form that can obscure the surgeon's view. Laser (for example, Q-switched Neodynium (Nd) YAG lasers, excimer laser to treat glaucoma, or an Erbium YAG laser) could be used to cut an opening through trabecular meshwork for releasing elevated intraocular pressure.

Recently, Ho:YAG lasers were used to generate a pulsed water-jet knife that minimized the amount of water flowing into the incision. Unlike the Nd:YAG laser, the wavelength of the Ho:YAG laser is about 2.1 μm, which is very close to the 1.9 μm peak absorption of water, and is absorbed about 100 times better than the light from an Nd:YAG laser. Further, Ho:YAG laser energy is reported to be uniformly absorbed by water-bearing tissue, irrespective of its pigmentation.

The laser was used to vaporize water confined in a small space of an instrument, such as in a catheter, a cannula, or a syringe-type delivery apparatus. The laser energy is absorbed by the water, which creates a localized vapor bubble that expands and ejects a pulse of water from the end of the instrument. Increasing the energy of the laser pulses creates higher-pressure water jets, as is known by those of ordinary skill in the art. By ways of illustration, a standard 1-mm diameter catheter or needle with a 5-mm long, 100-μm wide nozzle portion may hold a 400-μm diameter optical fiber. A reservoir of cold saline solution is maintained at the distal portion of the catheter between the proximal end of the nozzle and the distal end of the optical fiber. The optical fiber transmits Ho-YAG laser energy to the saline solution, where it vaporizes a small volume of water and ejected microliter volumes through the nozzle.

A laser-induced liquid jet is initiated with the generation of the laser. The laser creates a plasma in the liquid, which can be water, causing a dissociation of molecules, production of non-condensible gases, and shock waves and high pressures, which can be as high as one-thousand atmospheres or more. Around the laser-generated bubble beyond the first few microseconds, the fluid mechanics surrounding the bubble are essentially incompressible, inviscid, and irrotational. The rapid expansion of the fluid from liquid to plasma or gas form creates a jet of fluid which can be directed through an opening in the distal portion of the jet instrument.

Ho-YAG laser pulses between about 250 mJ and about 700 mJ were used to examine the characteristics of the water jet. The water jet was used as a knife to cut the rabbit's brain ventricles. The water jet penetrated about a fraction of a millimeter with a clean cut in line with the increased incident laser pulse energy; the surrounding blood vessels were not disrupted. The unique feature of the water jet is that its strength can be changed instantly, depending on the properties of the target tissue. The pulsed water jet imparts little heat to surrounding tissues and delivers only a small volume of water to the cutting site.

U.S. Pat. No. 5,860,972 issued to Hoang, entitled “Method of Detection and Destruction of Urinary Calculi and Similar Structures”, the entire contents of which are incorporated herein by reference, discloses a method of detection and destruction of urinary calculi comprising the steps of providing a laser source and a fiber optic laser delivery device, initiating laser energy transmission from the laser source and delivering an initial pulse of laser radiation to the urinary calculi for detection, generating and continuing to deliver pulses of laser energy to the urinary calculi until the urinary calculi has been fragmented completely, wherein the laser source may be a Ho:YAG laser.

US Patent Application publication 2004/0020905, entitled “Method and Apparatus for Cleaning Surfaces”, the entire contents of which are incorporated herein by reference, discloses a method for cleaning surfaces, the method comprises securing a surface to be cleaned in a liquid, focusing a laser beam at a point in the liquid to generate a liquid jet and a shock wave, and positioning the point of focus of the laser beam in close proximity to the surface to be cleaned such that the laser-induced liquid jet and shock wave clean the surface.

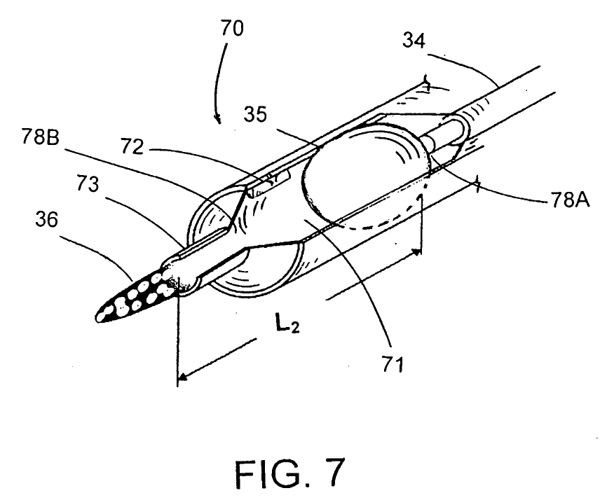

FIG. 7 shows an instrument 70 that may be used as a laser-induced liquid jet instrument. The liquid jet can be generated by a laser pulse being directed through an optic fiber 34 to saline solution inside an enclosure 71. The enclosure 71 is confined within the lumen of a catheter or hollow applicator 77 between the distal end 78A of an optical fiber 34 and the proximal end 78B of a nozzle 73. The laser energy vaporizes a small volume or a small portion of water and ejects, for example, about a microliter volume of water or water/bubble mixture 35 as a laser-induced liquid jet 36 through the nozzle 73. Of course, other volumes can be used, as is known by those of ordinary skill in the art. In one embodiment, the standoff distance, L₂, is between about 0.1 to 5 millimeters, preferably between about 0.3 to 1 millimeter for optimal jet characteristics. The liquid in the enclosure 71 may come from an external source 79. In one embodiment, the liquid in the enclosure 71 comes from the anterior chamber through a one-way check valve 72. In another embodiment, the liquid in the enclosure comes from the nozzle. In a further embodiment, the nozzle 73 may comprise a one-way flap structure for allowing jet bursting out of the enclosure. The enclosure may further comprise a pressure sensor for monitoring the pressure or pressure history of the fluid in the enclosure and optionally uses the monitored data for feedback control.

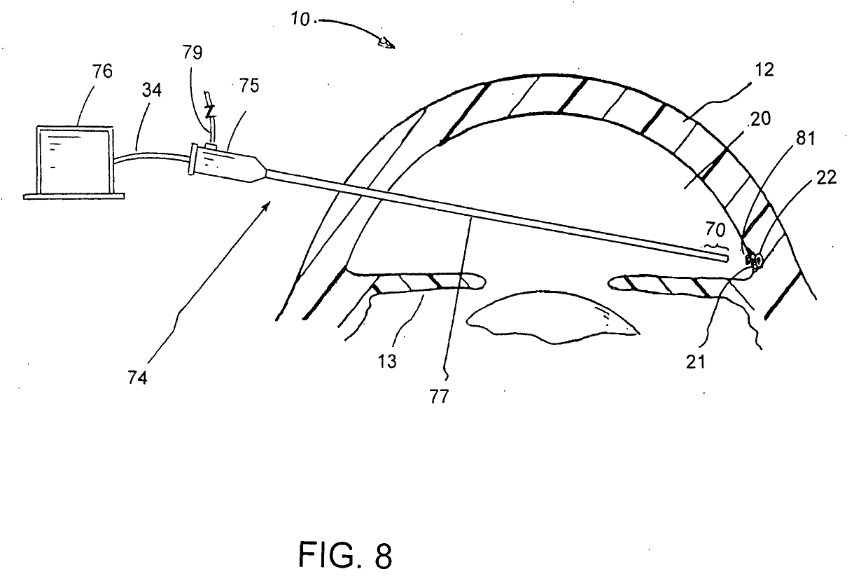

FIG. 8 shows a fiber optic probe system 74 for providing laser-induced liquid jet in an eye, wherein the distal portion can comprise the instrument 70 as described in FIG. 7. The fiber optic probe system 74 comprises a handle 75 connected to a catheter body 77, wherein a fiber optic 34 is mounted therethrough. The fiber optic 34 is further connected to a light source 76 for providing appropriate laser light to the instrument 70.

Some aspects of the disclosure relate to a method of treating glaucoma including the steps of providing a laser-induced liquid jet instrument, wherein the instrument comprises a laser energy source, an optical fiber means for transmitting laser beam, a small volume of liquid, and a distal end with an opening. The method further includes inserting the distal end of the instrument into an eye through a corneal incision and forwarding the opening against trabecular meshwork. The method also includes applying a pulse of laser beam to generate pulsed liquid jet or bubble jet for creating a hole at the trabecular meshwork. In one embodiment, the small volume of liquid comes from aqueous in the anterior chamber.

Some aspects of the disclosure relate to a method of treating glaucoma, which includes providing a laser-induced liquid jet instrument, wherein the instrument comprises a laser energy source, an optical fiber means for transmitting laser beam, a small volume of liquid, and a sharp distal end with an opening. The method includes inserting the distal end of the instrument into an eye through a corneal incision and advancing the sharp distal end to pass trabecular meshwork and place the opening at about Schlemm's canal. The method further includes applying a pulse of laser beam to generate pulsed liquid, liquid/bubble mixed jet, or shock wave for creating pulsed elevated pressure inside Schlemm's canal. In some further embodiments, the method further comprises placing a trabecular stent with a lumen in a prior step. The forwarding step can include advancing the distal end of the instrument through the lumen of the stent for positioning the opening at about Schlemm's canal.

Some aspects of the disclosure relate to a method of treating glaucoma. The method can include providing a laser-induced liquid jet instrument, wherein the instrument comprises a laser energy source, an optical fiber means for transmitting laser beam, a small volume of liquid, and a sharp distal end with an opening. The method also preferably includes, inserting the distal end of the instrument into an eye through a scleral incision ab externally and advancing the sharp distal end to position the opening at about Schlemm's canal. The method also includes applying a pulse of laser beam to generate pulsed liquid, liquid/bubble mixed jet, or shock wave for creating pulsed elevated pressure inside Schlemm's canal. In other embodiments, the fluid is at an elevated pressure effective to cause plastic deformation for at least a portion of the aqueous cavity.

Some aspects of the disclosure relate to a method of treating glaucoma, which includes inserting a stent through an incision in an eye, the stent having an inflow portion that is in fluid communication with an outflow portion of the stent, and transporting the stent from the incision through the anterior chamber of the eye to an aqueous cavity of the eye, such that the inflow portion of the stent is positioned in the anterior chamber and the outflow portion of the stent-is positioned at the aqueous cavity. The method further includes infusing fluid from the inflow portion to the outflow portion of the stent, wherein the fluid is a laser-induced liquid or liquid/bubble mixed jet. In some embodiments, the method can comprise a pulse duration in the range from 1 nanosecond to 100 microseconds. In further embodiments, the laser energy source may be selected from a group consisting of a YAG laser (for example, Ho:YAG laser, Nd:YAG laser, or Er:YAG laser), an excimer laser and CO₂ laser. In further embodiments, the laser fluency of the laser beam is in the range of about 0.5 J/cm² to about 100 J/cm². In further embodiments, the laser beam has a wavelength in the range from about 157 nm to about 10.6 μm. In some further embodiments, the laser beam has a frequency range from about 1 Hz to about 10 kHz.

Although preferred embodiments of the disclosure have been described in detail, certain variations and modifications will be apparent to those skilled in the art, including embodiments that do not provide all of the features and benefits described herein. Accordingly, the scope of the present disclosure is not to be limited by the illustrations or the foregoing descriptions thereof.

What is claimed is:

1. A method of treating glaucoma, comprising:
inserting a jet instrument into an anterior chamber of an eye through an incision in the eye;
advancing the jet instrument through the anterior chamber toward a trabecular meshwork of the eye;
generating with the jet instrument at least one of a liquid jet, plasma jet, and gas jet; and
creating a hole in the trabecular meshwork with said at least one of said liquid jet, plasma jet, and gas jet.

2. The method of claim 1, further comprising contacting the trabecular meshwork with the jet instrument before creating the hole.

3. The method of claim 1, wherein the liquid jet is generated using a laser.

4. The method of claim 1, wherein the liquid jet comprises water.

5. The method of claim 1, wherein said liquid jet comprises aqueous humor.

6. The method of claim 1, further comprising administering a therapeutic agent through the jet instrument.

7. A method of treating glaucoma, comprising:
inserting a distal portion of a jet instrument into an aqueous cavity of an eye;
generating at least one of a liquid jet, plasma jet, and gas jet with said jet instrument; and
introducing said at least one of a liquid jet, plasma jet, and gas jet into said aqueous cavity thereby increasing a fluid pressure in the aqueous cavity of the eye.

8. The method of claim 7, wherein the aqueous cavity comprises Schlemm's canal of the eye.

9. The method of claim 7, wherein the aqueous cavity comprises an aqueous collector channel of the eye.

10. The method of claim 7, wherein the aqueous cavity comprises an episcleral vein of the eye.

11. The method of claim 7, further comprising engaging the distal portion of the instrument with a glaucoma implant, said implant configured to conduct fluid away from the anterior chamber.

12. The method of claim 7, further comprising advancing said glaucoma implant with the distal portion of the instrument.

13. The method of claim 12, further comprising directing at least one of said liquid jet, plasma jet, and gas jet through a lumen of said implant, said implant being configured to conduct fluid away from the anterior chamber.

14. The method of claim 7, further comprising elevating said pressure to a level sufficient to cause plastic deformation of the aqueous cavity.

15. The method of claim 7, wherein said liquid jet comprises water.

16. The method of claim 7, wherein said liquid jet comprises aqueous humor.

17. The method of claim 7, further comprising administering a therapeutic agent through the jet instrument.

18. The method of claim 7, wherein the liquid jet is generated using a laser.
